(19) United States
(12) Patent Application Publication
(10) Pub. No.: US 2025/0131576 A1
(43) Pub. Date: Apr. 24, 2025

(54) PUPIL DETECTION METHODS IN OPHTHALMIC LASER SURGERY FOR DETECTING PUPIL IN EYE IMAGES CAPTURED BEFORE AND AFTER EYE APPLANATION

(71) Applicant: AMO Development, LLC, Irvine, CA (US)

(72) Inventor: Chaohong Wu, Fremont, CA (US)

(21) Appl. No.: 18/923,351

(22) Filed: Oct. 22, 2024

Related U.S. Application Data
(60) Provisional application No. 63/592,901, filed on Oct. 24, 2023.

(51) Int. Cl. G06T 7/13; A61F 9/008; G06T 5/30

(52) U.S. Cl. CPC G06T 7/13; A61F 9/008; G06T 5/30; G06T 2207/10024; G06T 2207/10048; G06T 2207/20032; G06T 2207/30041

(57) ABSTRACT

Two pupil detection methods implemented in an ophthalmic laser surgery system for detecting the pupil of the patient's eye both before and after the eye is applanated by a patient interface device. In the first method, an infrared image is captured before the eye is coupled to the patient interface. After excluding certain image artifacts, the pixel intensity is clustered into four clusters, and pixels belonging to the cluster with the lowest intensity are deemed pupil pixels, and pupil boundary and pupil center are determined accordingly. In the second method, a color image is captured after the eye is coupled to the patient interface. The color image is converted to the HSV color space, and only the hue channel is used for pupil detection. The hue channel image is filtered by median filtering and edge-preserving filtering (e.g. guided image filtering), then binarized, before pupil detection is performed.

### CROSS-REFERENCE TO RELATED APPLICATIONS

The present application claims priority to U.S. Provisional Application No. 63/592,901, filed on Oct. 24, 2023, the contents of which are expressly incorporated herein by reference.

### BACKGROUND OF THE INVENTION

This invention relates to ophthalmic laser surgery methods and related systems, and in particular, it relates to a method and system for pupil detection for ophthalmic laser surgery.

Ophthalmic laser systems, such as femtosecond laser systems, are used in various corneal refractive surgeries to make incisions in the cornea, such as to form corneal flaps or corneal lenticules. Prior to forming the incisions, the center of the cornea must be determined. This step, sometimes referred to as centration, typically include identifying the treatment center for lenticule cutting, as well as correcting cyclotorsion angle if astigmatism correction is included in the treatment. Proper centration requires accurate detection of the pupil in eye images. However, many factors make pupil detection challenging. For example, during the laser treatment, the eye is typically coupled to the ophthalmic laser system by a mechanical device referred to as a patient interface, and such coupling changes the shape of the eye. Some patient interfaces applanate the cornea, causing flattening of the cornea and distortion of the pupil. These changes present challenges for accurate pupil detection before and after applanation.

Some centration methods are known. For example, one method detects the centroid in infrared (IR) images of the eye captured before and after applanation. Some methods further apply corneal markers (e.g. ink marks) on the eye before applanation, and then detect the markers after applanation in order to determine cyclotorsion.

Some prior pupil detection methods are based on gradient computation. After the gradient information is computed, it is segmented with a heuristic threshold to get candidate edge pixels. An ellipse is fit to each candidate, and the candidate is evaluated based on metrics such as its ellipse aspect ratio, the angular spread of its edges relative to the ellipse, and the ratio of ellipse outline points that support the hypothesis of it being a pupil. This evaluation yields a confidence measure for each candidate to be the pupil, and the candidate with the highest confidence measure is then selected as detected pupil.

In some other pupil detection methods, the eye image is first convolved with a circular mask of a fixed-radius, followed by smoothing with average kernel, image contrast enhancement, gradient computation, gray-level conversion and index normalization, then basic Circular Hough Transform (CHT) is used to detect all the features of a circular shape, the position of the center and the radius.

However, many existing pupil detection and marker detection methods are unsatisfactory due to low accuracy.

### SUMMARY OF THE INVENTION

Accordingly, the present invention is directed to a method for pupil detection from images of the eye that substantially obviates one or more of the problems due to limitations and disadvantages of the related art.

An object of the present invention is to automatically and accurately detect the pupil in eye images captured both before and after corneal applanation.

Additional features and advantages of the invention will be set forth in the descriptions that follow and in part will be apparent from the description, or may be learned by practice of the invention. The objectives and other advantages of the invention will be realized and attained by the structure particularly pointed out in the written description and claims thereof as well as the appended drawings.

To achieve the above objects, the present invention provides a method for detecting a pupil of an eye, which includes: obtaining an infrared image of the eye; clustering pixel intensity values of the image to classify the pixel intensity values into four clusters; and determining a pupil boundary and a pupil center of the eye based on locations of all pixels that belong to a selected one of the four clusters.

In another aspect, the present invention provides a method for detecting a pupil of an eye, which includes: obtaining a color image of the eye, the color image being represented in an RGB color space; converting the color image into an HSV color space representation having a hue channel, a saturation channel, and a value channel, to obtain a hue channel image; filtering the hue channel image to obtain a filtered hue channel image; and detecting a pupil boundary and a pupil center of the eye based on the filtered hue channel image.

In another aspect, the present invention provides an ophthalmic laser system for treating a patient's eye, which includes: a laser source configured to generate a pulsed laser beam; an optical delivery system coupled to the laser source, configured to receive and direct the pulsed laser beam; a camera coupled to the optical delivery system, configured to obtain images of the eye; and a processor coupled to the laser source, the optical delivery system, and the camera, configured to perform the above methods for detecting a pupil of the eye.

It is to be understood that both the foregoing general description and the following detailed description are exemplary and explanatory and are intended to provide further explanation of the invention as claimed.

### BRIEF DESCRIPTION OF DRAWINGS

FIG. 1 schematically illustrates a method according to an embodiment of the present invention for automatically detecting the pupil in an eye image captured before the eye is coupled to the ophthalmic laser system by a patient interface.

FIGS. 2A and 2B respectively illustrate an exemplary IR image of an eye before applanation and the result of pupil detection using the method of FIG. 1.

FIG. 2C is histogram showing the number of pixels vs. pixel value for an exemplary IR eye image, and FIGS. 2D-2G show clustering result of the exemplary IR eye image, showing the fours clusters of pupil, iris, sclera, and near-saturated regions, respectively.

FIG. 3 schematically illustrates a method according to an embodiment of the present invention for automatically detecting the pupil in an image captured after the eye is coupled to the ophthalmic laser system by a patient interface and applanated.

FIGS. 4A-4D illustrate an exemplary RGB image of an eye captured after applanation, a corresponding hue channel image, a blurred hue channel image, and the result of pupil detection using the method of FIG. 3, respectively.

FIG. 5 schematically illustrates an ophthalmic laser system in which embodiments of the present invention may be implemented.

### DETAILED DESCRIPTION OF THE INVENTION

Embodiments of the present invention provide two pupil detection methods for detecting the pupil in eye images captured before and after applanation by the patient interface of the ophthalmic laser system, respectively.

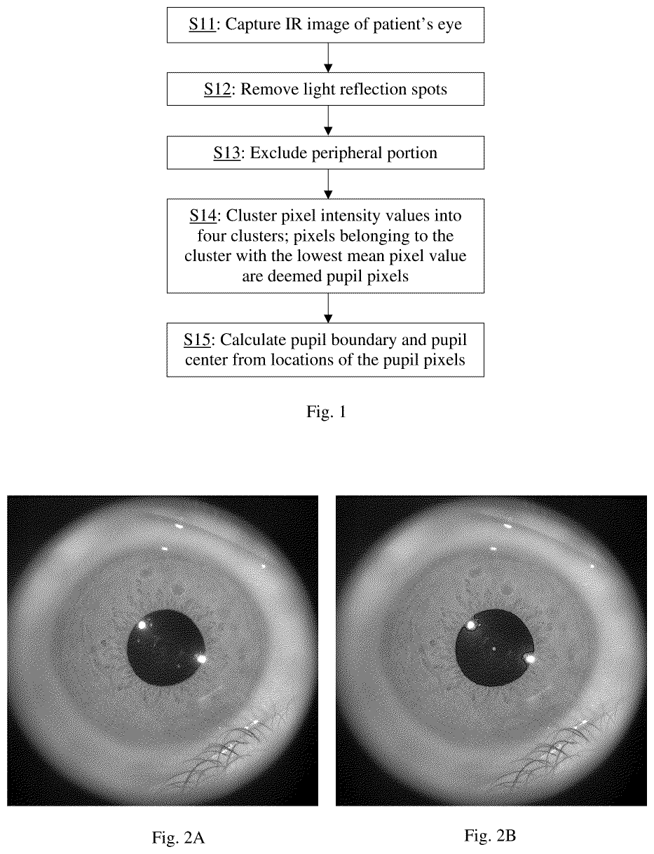

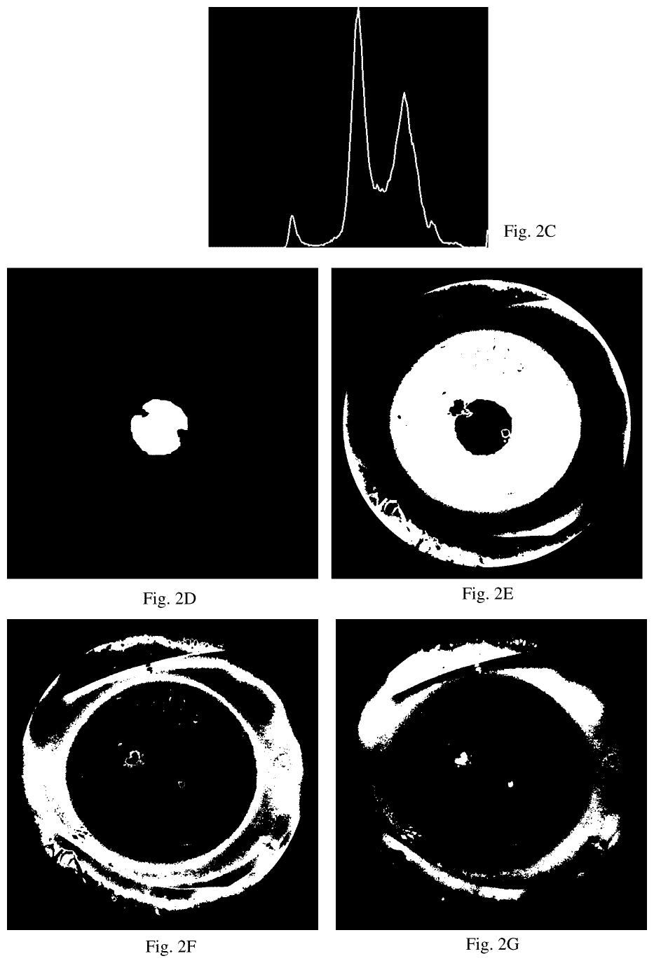

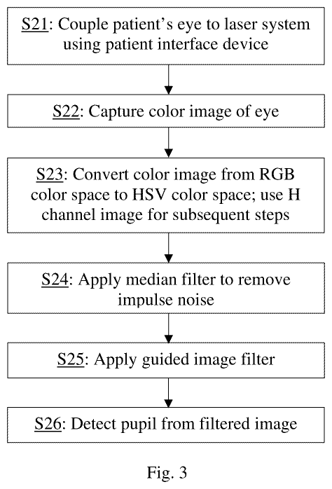

FIG. 1 schematically illustrates a method for detecting the pupil in an eye image captured before the eye is coupled to the ophthalmic laser system by a patient interface. The eye is in a free state and not applanated. The eye image is an IR image captured using an IR camera of the ophthalmic laser system (step S11). IR images are preferred to visible light images for this pupil detection method because dark pupils tend to produce higher quality image when IR images are used. FIG. 2A shows an exemplary IR image of an eye before applanation.

The captured IR image (a grayscale image) typically includes reflection spots generated by illumination lights of the imaging device. These reflection spots are generally small, isolated, and have high pixel intensities. The captured image is processed to remove such light reflection spots (step S12). More specifically, in a preferred embodiment, regions of the image having near-saturation intensities are identified using a threshold pixel value, for example, 235 for an 8-bit image. For each of these identified high intensity regions, dilation is performed using a small two-dimensional element (e.g., a circular structure, 1 to 7 pixels in size) to produce a larger region (the dilated region), and the average pixel value within the dilated stripe (i.e. area outside of the high intensity region and inside the dilated region) is used to replace the pixel values in the high intensity region.

In an alternative embodiment, rather than replacing their intensity values, pixels with intensities exceeding the threshold value are simply excluded from subsequent processing steps.

Note that step S12 may be omitted, in particular, if the image capture device setup is such that the IR image does not have light reflection spots.

Peripheral portions of the image that extend outside of the eye are excluded, i.e., only the remaining portion is selected for processing in the subsequent steps (step S13). For example, a radius may be set as an input parameter and the portion outside of the radius is excluded. Preferably, the radius of exclusion is just inside the outer boundary of the sclera in the image. Note that if the image is not originally centered on the eye, a preliminary step may be carried out to estimate the location and size of the eye in the image.

Note that step S13 may be omitted if the image capture device setup is such that the IR image only includes the eye, or the IR image has been pre-processed to only include the eye. Also, the sequence of performing steps S12 and S13 may be reversed.

Then, a clustering method is applied to the collection of pixel intensity values of the pixels of the image to classify the pixel values into four clusters, and pixels belonging to the cluster with the lowest mean pixel value (i.e. darkest) are deemed pupil pixels (step S14). Generally speaking, clustering is a data analysis technique that groups a set of objects (e.g., values) such that objects in the same cluster are more similar to each other than to those in other clusters. In a preferred embodiments, a K-Means clustering algorithm is used in this step. The number of clusters are set to four based on the empirical observation that there tends to be four main types of structures of different intensities in such eye images (after the peripheral portion, which have the darkest pixels, have been excluded in step S13): the pupil (the darkest pixels), the iris and some darker parts of the sclera, the sclera, and the near saturated regions from the illuminating lighting and some lighter parts of the sclera. Setting the number of clusters to three or fewer may degrade the pupil detection accuracy. Setting the number of clusters to five or more may cause the pupil pixels to be divided among two clusters. It is noted that there are algorithms that are able to decide the best number of clusters for a clustering method, but such algorithms often need large amounts of data to be trained.

An exemplary clustering result is shown in FIGS. 2D-2G, showing the fours clusters of pupil, iris, sclera, and near-saturated regions, respectively. FIG. 2C is a histogram showing the number of pixels vs. pixel value for this example. It can be seen that the pupil cluster is well separated from the other clusters.

In some embodiments, an OpenCV K-Means clustering program may be used in the clustering step. In one example, the iteration termination criteria and other parameters inputted to the program are set as follows:
The type of termination criteria: TermCriteria::EPS+TermCriteria::COUNT
The maximum number of iterations or elements to compute: 10
The desired accuracy or change in parameters at which the iterative algorithm stops at: 1.0
Attempts: 5
Center initialization: the flag is set to KMEANS_PP_CENTERS, which adopts k-means++center initialization described in D. Arthur and S. Vassilvitskii, “k-means++: The Advantages of Careful Seeding,” SODA '07: Proceedings of the Eighteenth Annual ACM-SIAM Symposium on Discrete Algorithms, January 2007, Pages 1027-1035.

Other parameters, and other suitable programs, may alternatively be used to perform the clustering step.

The output of the clustering step is a cluster index for each pixel of the image. As mentioned earlier, pixels belonging to the cluster with the lowest mean pixel value are deemed pupil pixels. The pupil boundary and pupil center are then determined based on the locations of the pupil pixels (step S15). As there may be small blobs in the pupil cluster in addition to the pupil, small contours are eliminated by setting a threshold so that only a larger sized contour (there should be only one large contour in the pupil segment image) or the largest contour is identified as the pupil boundary. In one embodiment, the pupil boundary is the boundary of the image region formed by the greatest number of pupil pixels that are connected to each other, and the pupil center is the centroid of such image region. Image moments, which are particular weighted averages of image pixel intensities, may be used to find specific properties of an image such as radius, area, centroid etc. The centroid is given by the formula: Cₓ=M₁₀/M₀₀, C_{y}=M₀₁/M₀₀, where Cₓ and Cₓ are the x and y coordinates of the centroid and M₁₀, M₀₁ and M₀₀ are three moments. In some embodiments, image region formed by the pupil pixels may be fitted to a circle or an ellipse to determine the pupil boundary, and the center of the circle or ellipse may be calculated as the pupil center.

FIG. 2B illustrates the result of pupil detection for the IR image in FIG. 2A using the above-described method. In FIG. 2B, the pink line indicates the detected pupil boundary and the green dot indicates the detected pupil center, overlayed on the original IR image.

The clustering-based pupil detection method described above can accommodate variations in illumination as well as various eye types of different races, and can detect pupils with high accuracy in all such situations. Moreover, no hard thresholding is needed to segment the pupil from the other eye structures including the iris, the sclera, and the cornea.

After the eye is coupled to the ophthalmic laser system by the patient interface and applanated, pupil detection is critical a step in detecting markers previously formed on the cornea, where the radius and centroid of the pupil is used as the basis for detecting the previously formed corneal markers. For example, three markers may have been formed in the top, left and right regions of the sclera surrounding the pupil. The appearance of the eye, however, is much different after applanation. Therefore, a different method is used to detect the pupil after applanation, as described below with reference to FIG. 2.

After coupling the patient interface to the eye (applanation) (step S21), a color image of the eye is captured through the optical components of the patient interface using a visible-light camera of the ophthalmic laser system (step S22). Typically, visible-light cameras produce color images in the RGB (red, green, blue) color space. The color image of the eye is then converted from the RGB color space to the HSV color space (step S23), where colors are represented by three components: Hue (dominant wavelength), Saturation (purity/shades of the color), and Value (intensity). In the HSV color space, only the hue channel describes color information, which makes it the ideal feature space for pupil segmentation and detection for this application. The inventor has tested using other color spaces, such as LAB and YCrCb, and the HSV color space yielded the best pupil detection results. The subsequent steps are performed only on the hue channel image.

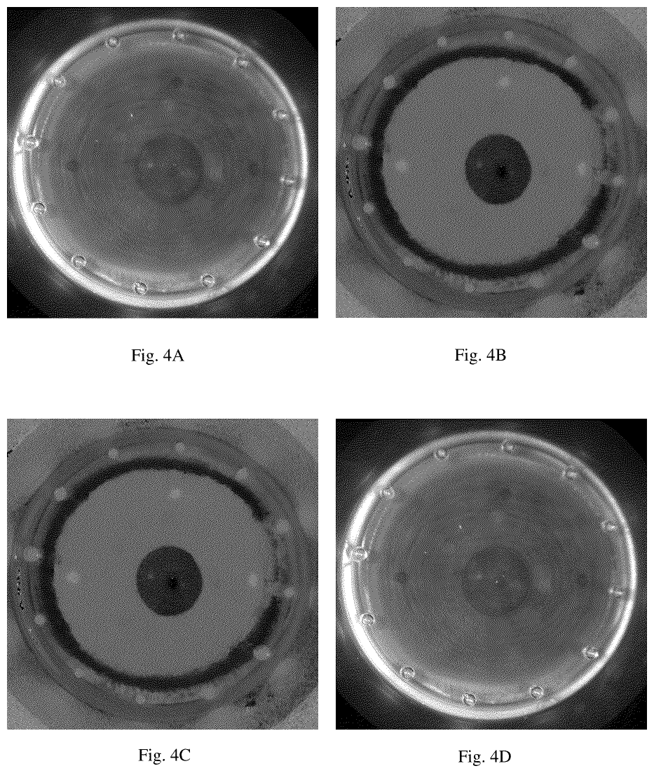

FIG. 4A shows an exemplary RGB image of an eye after applanation, and FIG. 4B shows the corresponding hue channel image.

As salt-and-pepper noise (impulse noise) are typically present in the hue channel, a median filter (median blurring) is applied to filter out noisy pixels (step S24). The size of the median filter may be, for example, 3×3 or 5×5 pixels. FIG. 4C shows the median blurred hue channel image.

Then, an edge-preserving filter, such as a guided image filter, is applied to the median blurred hue channel image (step S25), which helps to improve pupil detection accuracy and to produce a clear boundary. The diameter of the pixel neighborhood used in the guided image filtering, i.e., the Sigma space parameter for spatial extent of the kernel, controls the filtering effect in both the x and y spatial directions. Edge-preserving filtering, such as guided image filtering, can selectively blur similar intensity pixels in the spatial neighborhoods, while preserving sharp edges. In guided image filtering, (1) pixels of similar intensities, and are spatially near the filtered pixel, will have influence on the output; (2) pixels that are spatially far away from the filtered pixel will have little influence (due to the spatial kernel); and (3) pixels that have dissimilar intensities will have little influence (due to the intensity kernel), even if they are spatially close to the filtered pixel.

The effects of the median filtering and guided image filtering steps enable the subsequent edge detection (pupil detection) step to locate the pupil accurately. Median filtering and guided image filtering are generally known in the art and their details are omitted here. OpenCV programs or other suitable programs may be used to perform these filtering steps.

After the filtering steps, the pupil is detected from the filtered image (step S26). More specifically, in one embodiment, the image is binarized to generate a binary image, for example, using Otsu's binarization method; the black region (i.e., image region formed of black pixels connected to each other) located at the center of the binary image is deemed the pupil, and its boundary and centroid are determined as the pupil boundary and pupil center, respectively. The black region may alternatively be fitted to a circle or an ellipse to determine the pupil boundary and pupil center. In alternative embodiments, edge or contour detection may be applied to detect edges in the filtered hue channel image, and the detected edges are fitted to a circle or an ellipse to determine the pupil boundary and pupil center. Hough transform circle detection may also be applied although the computation is more complex; RANSAC circle fitting may also be applied.

FIG. 4D shows the result of pupil detection using the above method. In FIG. 4D, the red circle indicates the detected pupil boundary and the green dot indicates the detected pupil center, overlayed on the original color image.

The above-described pupil detection methods may be applied in other ophthalmic laser surgeries, such as cataract procedures that also includes astigmatism correction. Moreover, pupil detection is a fundamental component of real-time eye tracking techniques, and the above-described pupil detection methods may find applications there.

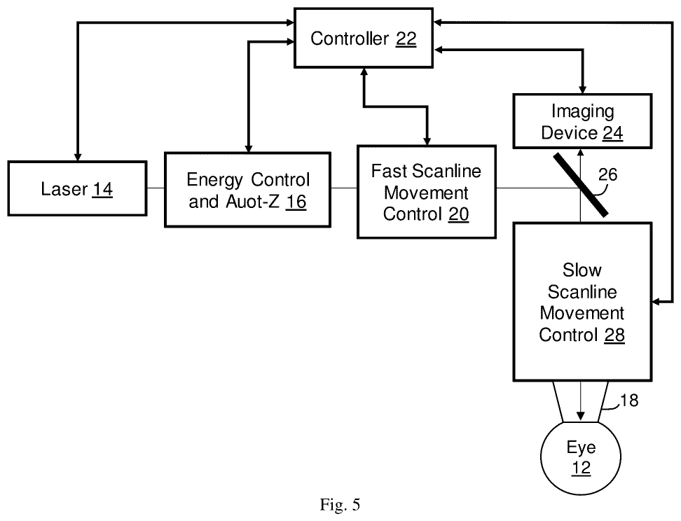

The above-described pupil detection methods may be implemented in a control computer of an ophthalmic surgical laser system. Many such laser systems are known. A block diagram of an exemplary ophthalmic laser system is shown in FIG. 5.

As shown in FIG. 5, the ophthalmic surgical laser system 10 is designed for making incisions in a tissue of a patient's eye 12. The system 10 includes, but is not limited to, a laser 14 capable of generating a pulsed laser beam, an energy control module 16 for varying the pulse energy of the pulsed laser beam, a fast scanline movement control module 20 for generating a fast scanline of the pulsed laser beam, a slow scanline movement control module 28 for moving the laser scanline and delivering it to the tissue 12, and a controller (control computer) 22. The fast scanline movement control module 20 and the slow scanline movement control module 28 collectively form an optical delivery system for delivering the pulsed laser beam to the eye tissue. The laser 14 may be a femtosecond laser capable of providing a pulsed laser beam, which interacts with the eye tissue to produce localized photodisruption (e.g., laser induced optical breakdown). Such localized photodisruptions can be placed at or below the surface of the tissue to produce high-precision processing such as incisions.

The system 10 further includes a beam splitter 26 and an imaging device (e.g., a video camera and/or an IR camera) 24 coupled to the controller 22 for imaging the eye before and/or during the surgery and to provide a feedback control mechanism of the pulsed laser beam. Additional imaging systems and feedback methods may also be used.

The laser system 10 also includes a patient interface device 18 for coupling the eye to the laser system to immobilize the eye relative to the optical delivery system prior to delivering the pulsed laser beam to the eye. The patient interface device 18 includes a lens that contacts and changes the shape (for example, flatten or curve) of the cornea.

The controller 22, such as a processor executing suitable control software, is operatively coupled with the other components to control the operation of the system, such as to direct the pulsed laser beam along a scan pattern to the eye tissue 12. The pupil detection method described above may be implemented by the control computer 22.

It will be apparent to those skilled in the art that various modification and variations can be made in the pupil detection method and related apparatus of the present invention without departing from the spirit or scope of the invention. Thus, it is intended that the present invention cover modifications and variations that come within the scope of the appended claims and their equivalents.

1. A method for detecting a pupil of an eye, comprising:
obtaining an infrared image of the eye;
clustering pixel intensity values of the image to classify the pixel intensity values into four clusters; and
determining a pupil boundary and a pupil center of the eye based on locations of all pixels that belong to a selected one of the four clusters.

2. The method of claim 1, wherein the step of obtaining the infrared image of the eye includes:
capturing an image of the eye using an infrared camera;
removing or excluding isolated high intensity regions which have pixel intensities exceeding a saturation threshold; and
excluding peripheral portions of the image that extend outside of the eye.

3. The method of claim 2, wherein the step of removing the isolated high intensity regions includes:
dilating each high intensity region to generate a dilated region;
for each high intensity region, calculating an average pixel intensity for a dilated stripe region outside of the high intensity region and inside the corresponding dilated region; and
replacing pixel intensities in each high intensity region with the average pixel intensity of the corresponding dilated stripe region.

4. The method of claim 1, wherein the step of clustering pixel intensity values uses k-means clustering.

5. The method of claim 1, wherein the selected cluster is one that has a lowest mean pixel value among the four clusters.

6. The method of claim 1, wherein the step of determining the pupil boundary and the pupil center of the eye includes:
determining a boundary of an image region formed by a greatest number of pixels belonging to the selected cluster and connected to each other; and
determining a centroid of the image region.

7. The method of claim 1, wherein the step of determining the pupil boundary and the pupil center of the eye includes:
fitting an image region formed by a greatest number of pixels belonging to the selected cluster and connected to each other to a circle or an ellipse; and
determining a center of the circle or the ellipse.

8. A method for detecting a pupil of an eye, comprising:
obtaining a color image of the eye, the color image being represented in an RGB color space;
converting the color image into an HSV color space representation having a hue channel, a saturation channel, and a value channel, to obtain a hue channel image;
filtering the hue channel image to obtain a filtered hue channel image; and
detecting a pupil boundary and a pupil center of the eye based on the filtered hue channel image.

9. The method of claim 8, wherein the step of obtaining the color image of the eye includes:
coupling the eye to an ophthalmic laser system by a patient interface device, wherein the patient interface device applanates a surface area of the eye; and
capturing the color image of the eye through the patient interface using a camera of the ophthalmic laser system.

10. The method of claim 8, wherein the filtering step includes median filtering the hue channel image.

11. The method of claim 10, wherein the filtering step further includes guided image filtering the hue channel image after the median filtering.

12. The method of claim 8, wherein the step of detecting the pupil boundary and the pupil center of the eye includes:
binarizing the filtered hue channel image to obtain a binary image;
determining a boundary of an image region formed by black pixels that are connected to each other and located in a center region of the binary image; and
determining a centroid of the image region.

13. The method of claim 8, wherein the step of determining the pupil boundary and the pupil center of the eye includes:
binarizing the filtered hue channel image to obtain a binary image;
fitting an image region formed by black pixels that are connected to each other and located in a center region of the image to a circle or an ellipse; and
determining a center of the circle or the ellipse.

14. An ophthalmic laser system for treating a patient's eye, comprising:
a laser source configured to generate a pulsed laser beam;
an optical delivery system coupled to the laser source, configured to receive and direct the pulsed laser beam;
an infrared camera coupled to the optical delivery system, configured to obtain an infrared image of the eye; and
a processor coupled to the laser source, the optical delivery system, and the infrared camera, configured to perform a process to detect a pupil of the eye, the process including:
clustering pixel intensity values of the image to classify the pixel intensity values into four clusters; and
determining a pupil boundary and a pupil center of the eye based on locations of all pixels that belong to a selected one of the four clusters.

15. The ophthalmic laser system of claim 14, wherein the process further includes:
removing or excluding isolated high intensity regions from the image which have pixel intensities exceeding a saturation threshold; and
excluding peripheral portions of the image that extend outside of the eye.

16. The ophthalmic laser system of claim 15, wherein the step of removing the isolated high intensity regions includes:
dilating each high intensity region to generate a dilated region;
for each high intensity region, calculating an average pixel intensity for a dilated stripe region outside of the high intensity region and inside the corresponding dilated region; and
replacing pixel intensities in each high intensity region with the average pixel intensity of the corresponding dilated stripe region.

17. The ophthalmic laser system of claim 14, wherein the step of clustering pixel intensity values uses k-means clustering.

18. The ophthalmic laser system of claim 14, wherein the selected cluster is one that has a lowest mean pixel value among the four clusters.

19. The ophthalmic laser system of claim 14, wherein the step of determining the pupil boundary and the pupil center of the eye includes:
determining a boundary of an image region formed by a greatest number of pixels belonging to the selected cluster and connected to each other; and
determining a centroid of the image region.

20. The ophthalmic laser system of claim 14, wherein the step of determining the pupil boundary and the pupil center of the eye includes:
fitting an image region formed by a greatest number of pixels belonging to the selected cluster and connected to each other to a circle or an ellipse; and
determining a center of the circle or the ellipse.

21. An ophthalmic laser system for treating a patient's eye, comprising:
a laser source configured to generate a pulsed laser beam;
an optical delivery system coupled to the laser source, configured to receive and direct the pulsed laser beam;
a camera coupled to the optical delivery system, configured to capture a color image of the eye, the color image being represented in an RGB color space; and
a processor coupled to the laser source, the optical delivery system, and the camera, configured to perform a process to detect a pupil of the eye, the process including:
converting the color image into an HSV color space representation having a hue channel, a saturation channel, and a value channel, to obtain a hue channel image;
filtering the hue channel image to obtain a filtered hue channel image; and
detecting a pupil boundary and a pupil center of the eye based on the filtered hue channel image.

22. The ophthalmic laser system of claim 21, wherein the color image of the eye is captured through a patient interface which has been coupled to the eye, wherein the patient interface device applanates a surface area of the eye using a camera of the ophthalmic laser system.

23. The ophthalmic laser system of claim 21, wherein the filtering step includes median filtering the hue channel image.

24. The ophthalmic laser system of claim 21, wherein the filtering step further includes guided image filtering the hue channel image after the median filtering.

25. The ophthalmic laser system of claim 21, wherein the step of detecting the pupil boundary and the pupil center of the eye includes:
binarizing the filtered hue channel image to obtain a binary image;
determining a boundary of an image region formed by black pixels that are connected to each other and located in a center region of the binary image; and
determining a centroid of the image region.

26. The ophthalmic laser system of claim 21, wherein the step of determining the pupil boundary and the pupil center of the eye includes:
binarizing the filtered hue channel image to obtain a binary image;
fitting an image region formed by black pixels that are connected to each other and located in a center region of the image to a circle or an ellipse; and
determining a center of the circle or the ellipse.
